(19) United States
(12) Patent Application Publication
(10) Pub. No.: US 2020/0114136 A1
(43) Pub. Date: Apr. 16, 2020

(54) INTEGUMENTAL DISSOLVING NEEDLES AND NEEDLE DEVICES

(71) Applicant: Norihiro NANGOU, Tokyo (JP)

(72) Inventor: Norihiro NANGOU, Tokyo (JP)

(21) Appl. No.: 16/714,821

(22) Filed: Dec. 16, 2019

Related U.S. Application Data
(63) Continuation of application No. PCT/JP2018/010912, filed on Mar. 19, 2018.

(30) Foreign Application Priority Data
Jun. 22, 2017 (JP) 2017-121980

(51) Int. Cl. A61M 37/00; A01N 25/12; A01G 7/06

(52) U.S. Cl. CPC A61M 37/00; A61M 2205/0238; A01G 7/06; A01N 25/12

(57) ABSTRACT

There currently exists no needle capable of delivering pharmaceutical or cosmetic ingredient(s) into deep layers of integumental tissue (e.g. skin, scales, bark), nor any needle device designed to facilitate the precise administration of a desired dose, and to limit inflammation, pain, and other side effects associated with the needle(s). The present invention relates to an integumental dissolving needle—which houses micronized cosmetic/pharmaceutical ingredient(s), encapsulated by a coating agent absorbed into the integument—arranged on the surface or surfaces of a poultice. Needle thickness and length may be varied according to the biological species of interest. In addition, dosage(s) is/are printed on each section of the device to make explicit how many milligrams of the ingredient are present per unit area; these sections may be separated from one another along groove(s) or other markers on the device. The present invention is a needle device consisting of the above components.

The contents of the following Japanese patent application(s) are incorporated herein by reference:
2017-121980 filed in JP on Jun. 22, 2017.
PCT/JP2018/010912 filed on Mar. 19, 2018

### BACKGROUND

### 1. Technical Field

The present invention relates to integumental dissolving needles capable of delivering pharmaceutical or cosmetic ingredients into deep layers of integumental tissue (e.g. skin, scales, bark); and needle devices incorporating them designed to facilitate the precise administration of a desired dose, and to limit inflammation, pain, and other side effects associated with their application.

### 2. Related Art

Conventional intradermal dissolving microneedles (“MNs”) such as those described in Patent Refs. 1 and 2 can deliver pharmaceutical or cosmetic ingredients into the upper layers of human skin (e.g. epidermis, stratum corneum), but are unable to reach the deepest layers of human skin. While large MN arrays employing a needle length of 800 such as that described in Non-Patent Ref. 1, can deliver pharmaceutical or cosmetic ingredients as deep as the human dermis, they cause pain in the skin after their application. Human skin ranges from 1-4 mm in thickness (Non-Patent Ref. 2); however, cow skin is 5-7 mm in thickness, and dog skin is exceedingly thin (Non-Patent Ref. 3). This variability requires users to select MN devices having a needle length suitable for the species of interest. Moreover, conventional MN arrays do not intuitively indicate how many milligrams of ingredient(s) are present in a given unit area, nor do they employ grooves or perforations to facilitate the sectioning of the array, nor are such arrays ‘pre-sectioned’ for sale. The absence of such elements makes it difficult to precisely administer a desired dose.

### RELATED ART DOCUMENTS

### Patent Literature

[Patent Reference 1] Published unexamined patent application 2010-82401. In Japanese.
[Patent Reference 2] Published unexamined patent application 2012-25723. In Japanese.

### Non Patent Literature

[Non-Patent Reference 1] Advanced Science, Technology & Management Research Institute of Kyoto. [2011 Strategic Foundational Technology Improvement Support Operation, R&D Report: Development of novel tip-loaded drug-delivery microneedles, and applications to hair growth formulations.] March 2012. In Japanese. http://www.chusho.meti.go.jp/keiei/sapoin/portal/seika/2010/22h-73.pdf
[Non-Patent Reference 2] Hisashi Ishihara. [The Structure of the Skin.] 10 Apr. 2015. In Japanese. http://www.ams.eng.osaka-u.ac.jp/user/ishihara/?p=432
[Non-Patent Reference 3]: Kaneko Mikihiro. [5. Learn How the Body Works, 30: Learning How the Skin Works (to Raise a Healthy Horse).] In Japanese. http://www.b-t-c.or.jp/btc_p300/btcn/btcn68/btcn068-04.pdf

### GENERAL DISCLOSURE

The present invention was developed to solve the following problems:
How to facilitate the delivery of a pharmaceutical or cosmetic ingredient of interest into the deepest layers of integumental tissue (e.g. skin, scales, bark);
How to limit subsequent inflammation, pain, and other side effects associated with needle device(s); and
How to facilitate the precise administration of a desired amount of ingredient(s) by indicating dosage or dosages in an intuitive way, i.e. how many milligrams of the ingredient(s) are present in a given unit area.

The present invention provides an integumental (e.g. skin, scales, bark) dissolving needle, which is filled with micronized pharmaceutical ingredient(s) or micronized cosmetic ingredient(s) encapsulated by a layer of coating agent (“coating layer”) that is absorbed into the integument (e.g. skin, scales, bark), to allow the said ingredient(s) to penetrate into deep layers of the integument (e.g. skin, scales, bark). Needle thickness and length may be varied according to the biological species of interest.

The present invention provides an integumental (e.g. skin, scales, bark) dissolving needle fabricated such that suitable pharmaceutical ingredient(s) or cosmetic ingredient(s) are an integral component of the needle itself, to directly administer the said ingredient(s) without needing to wait for the needle to dissolve. Needle thickness and length may be varied according to the biological species of interest.

The present invention provides an integumental (e.g. skin, scales, bark) dissolving needle fabricated such that suitable pharmaceutical ingredient(s) covered with a coating layer, or cosmetic ingredient(s) covered with a coating layer, are an integral component of the needle itself, to administer the said ingredient(s) (without needing to wait for the needle to dissolve) and to simplify the manufacturing process. Needle thickness and length may be varied according to the biological species of interest.

The present invention provides an integumental (e.g. skin, scales, bark) dissolving needle device, in which needle(s) are arranged on the application-side surface of a poultice or surfaces of a poultice (e.g. hot compress, cold compress, anti-inflammatory analgesic tape), to limit subsequent inflammation, pain, and other side effects associated with needle(s).

The present invention provides an integumental dissolving needle device, on which dosage or dosages is printed to clearly indicate how many milligrams of ingredient(s) are present in a given unit area, and which contains groove(s) or perforations in the said device to facilitate the precise administration of a desired dose. For example, an embodiment might facilitate the removal of 1 cm² unit(s), each containing 10 mg (or 1.25 mg, etc.) of a pharmaceutical ingredient, by sectioning the needle device into 1 cm² units by groove(s) or perforations, and having “10 mg” (“1.25 mg”, etc.) printed on each unit. These characteristics make it easier for a user to break, cut, or otherwise divide the device and administer the desired dose.

The present invention provides an integumental dissolving needle device, on which dosage or dosages is printed to clearly indicate how many milligrams of ingredient(s) are present in a given unit area, and which is pre-sectioned to facilitate the precise administration of a desired dose. For example, a possible embodiment is pre-sectioned, 1 cm² units, each containing 10 mg (or 1.25 mg) of a pharmaceutical ingredient, and on each of which is printed “10 mg” (or “1.25 mg”, etc.). One or more units could then be applied to administer the desired dose.

The integumental (e.g. skin, scales, bark) dissolving needle, filled with micronized pharmaceutical or cosmetic ingredient(s) encapsulated by a coating agent that is absorbed into the integument (e.g. skin, scales, bark), offers the beneficial effects of allowing the encapsulated granules to penetrate deep into the integument (e.g. skin, scales, bark) once the needle itself dissolves in the integument (e.g. skin, scales, bark). This design offers superior penetrability to conventional MN(s).

The integumental dissolving needle fabricated such that suitable pharmaceutical ingredient(s) or cosmetic ingredient(s) are an integral component of the needle itself, offers the beneficial effects of allowing the said ingredient(s) to be administered directly, without waiting for the needle to dissolve, thereby allowing the said ingredient(s) to quickly penetrate into the integument.

The integumental dissolving needle fabricated such that suitable pharmaceutical ingredient(s) encapsulated by a coating layer, or suitable cosmetic ingredient(s) encapsulated by a coating layer, are an integral component of the needle itself, offers the beneficial effects of allowing the said ingredient(s) to be administered (without needing to wait for the needle to dissolve), and to simplify the manufacturing process, thereby allowing the said ingredient(s) to quickly penetrate into the integument.

The integumental (e.g. skin, scales, bark) dissolving needle device, in which any of the needles described above are arranged on the application-side surface of a poultice or surfaces of a poultice (e.g. hot compress, cold compress, anti-inflammatory analgesic tape), offers the beneficial effects of minimizing subsequent inflammation, pain, and other side effects associated with the needle(s).

The integumental dissolving needle device, on which dosage or dosages is printed and which contains groove(s) or perforations, offers the beneficial effect of facilitating the precise administration of a desired dose. For example, an embodiment might facilitate the removal of 1 cm² unit(s), each containing 10 mg (or 1.25 mg, etc.) of a pharmaceutical ingredient, by sectioning the needle device into 1 cm² units by groove(s) or perforations, and having “10 mg” (“1.25 mg”, etc.) printed on each unit. These characteristics make it easier for a user to break, cut, or otherwise divide the device and administer the desired dose.

The integumental dissolving needle device, on which dosage or dosages is printed and which is pre-sectioned, offers the beneficial effect of facilitating the selection of a desired dose. For example, a possible embodiment has pre-sectioned, 1 cm² units, each containing 10 mg (or 1.25 mg) of a pharmaceutical ingredient, and each is printed with “10 mg” (“1.25 mg”, etc.). One or more units could then be applied to administer the desired dose.

### BRIEF DESCRIPTION OF THE DRAWINGS

FIG. 1. Integumental dissolving needle device with dosage printed on surface (groove type; perspective view)

FIG. 2. Integumental dissolving needle device with dosage printed on surface (perforation type; perspective view)

FIG. 3. Integumental dissolving needle device with dosage printed on surface(pre-sectioned type; perspective view)

FIG. 4. Needle device housing cosmetic or pharmaceutical ingredient(s), micronized to a diameter on the micrometer order or smaller, covered with a layer of coating agent that is absorbed into the integument (cross-section view).

FIG. 5. Needle device housing granules of different layer structures. In principle, granules may have any plural number of layers. The figure depicts a specific embodiment containing: two-layer granules, consisting of cosmetic or pharmaceutical ingredient(s), micronized to a diameter on the micrometer order or smaller, encapsulated by a layer of coating agent that is absorbed into the integument; as well as four-layer granules, consisting of the said (two-layer) granules further covered with a layer of micronized cosmetic or pharmaceutical ingredient(s), followed by another layer of coating agent (cross-section view).

FIG. 6. Needle device housing multi-layer granules consisting of cosmetic or pharmaceutical ingredient(s) micronized to a diameter on the micrometer order or smaller, which are encapsulated by a layer of coating agent that is absorbed into the integument, which is further covered with a layer of micronized cosmetic or pharmaceutical ingredient(s), followed by another layer of coating agent (cross-section view).

### DESCRIPTION OF EXEMPLARY EMBODIMENTS

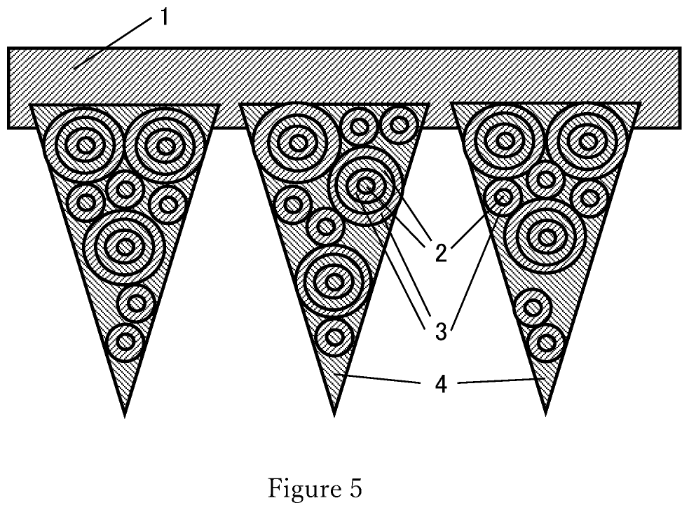

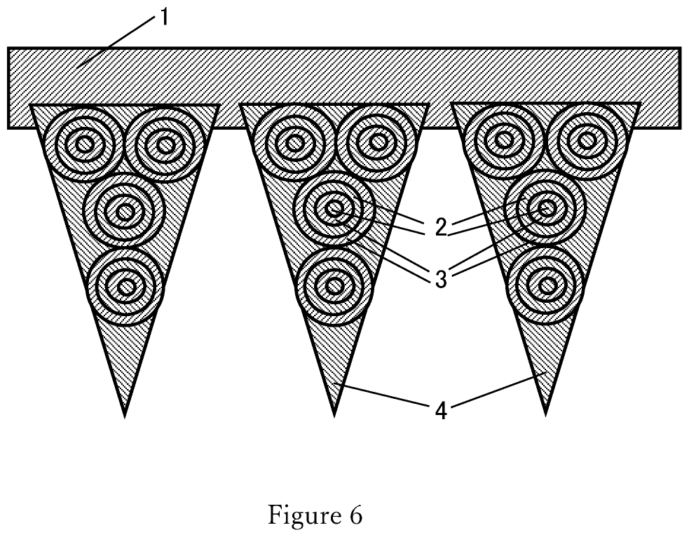

In one embodiment, a cosmetic ingredient 2 micronized to a diameter on the micrometer order or smaller (or similarly micronized pharmaceutical ingredient 2) is encapsulated by a layer of coating agent 3 that is absorbed into the integument (e.g. skin, scales, bark). These granules are housed in an integumental (e.g. skin, scales, bark) dissolving needle 4. Granules may possess more than two layers: FIGS. 5 and 6 depict four-layer granules, composed of the two-layer granules described immediately above, further coated with an additional layer of micronized cosmetic (or pharmaceutical) ingredient 2, followed by an additional layer of coating agent 3. Some such capsules depicted in FIG. 5, and all depicted in FIG. 6, have a four-layer structure; however, granules of more than four layers are possible. Needle 4 thickness and length may be varied according to the biological species of interest.

If possible, the integumental dissolving needle 4 may be fabricated such that the pharmaceutical ingredient 2 (or cosmetic ingredient 2, or coated pharmaceutical ingredient 2, or coated cosmetic ingredient 2) is an integral component of the needle itself. In this case, the coated pharmaceutical or cosmetic ingredient 2 merely housed in the needle 4 may differ from the cosmetic ingredient 2 (or pharmaceutical ingredient 2, or coated pharmaceutical ingredient 2, or coated cosmetic ingredient 2) present in the needle's composition. For example, a needle 4 might house an encapsulated hypertension drug, while compositionally containing an antibacterial agent. Needle 4 thickness and length may be varied according to the biological species of interest.

In another embodiment, the needle(s) 4 described above may be arranged on the application-side surface of a poultice 1 or surfaces of a poultice 1 (e.g. hot compress, cold compress, anti-inflammatory analgesic tape). Alternatively, the needle(s) 4 may be arranged on a patch 1 if anti-inflammatory drug-containing poultices cannot be used (e.g. if the device is for use by a person (or species) allergic to an anti-inflammatory drug or analgesic, or a person (or species) that does not respond to the anti-inflammatory drug or analgesic).

The composition of the coating agent 3 shall include at least one of the following biocompatible substances: nucleic acid esters, nucleotides, phosphate, polylactic acid salts, polylactic acid esters (including polylactic acid and polyglycolic acid copolymers), saccharides (including mucopolysaccharides [e.g. hyaluronic acid], dextran, maltose, glucose, sucrose, galactose, lactose, cellulose, carboxymethyl cellulose, hydroxyethyl cellulose, trehalose, peptidoglycans, polyglycolic acid, and chitin), amino acid esters, amino acid salts, proteins (e.g. gelatin, collagen, keratin), biodegradable polymers (e.g. peptides, lignin, polyvinyl alcohol, polyvinyl pyrrolidone), fullerene, vitamins, hormones, antigens, antibodies, substrates, and enzymes; alternatively or additionally, derivatives of any of these substances, and/or some mixture of them. The substances above are given as examples: any biocompatible substance capable of encapsulating the cosmetic or pharmaceutical ingredient, and being absorbed into the integument (e.g. skin, scales, bark) of the species of interest, may be used as (or in) the coating agent.

The composition of the aforementioned integumental dissolving needle 4 shall include at least one of the following biocompatible substances: nucleic acid esters, nucleotides, phosphate, polylactic acid salts, polylactic acid esters (including polylactic acid and polyglycolic acid copolymers), saccharides (including mucopolysaccharides [e.g. hyaluronic acid], dextran, maltose, glucose, sucrose, galactose, lactose, cellulose, carboxymethyl cellulose, hydroxyethyl cellulose, trehalose, peptidoglycans, polyglycolic acid, and chitin), amino acid esters, amino acid salts, proteins (e.g. gelatin, collagen, keratin), biodegradable polymers (e.g. peptides, lignin, polyvinyl alcohol, polyvinyl pyrrolidone), fullerene, vitamins, hormones, antigens, antibodies, substrates, and enzymes; alternatively or additionally, derivatives of any of these substances, and/or some mixture of them. The substances above are given as examples: any biocompatible substance capable of composing the needle, and being absorbed into the integument (e.g. skin, scales, bark) of the species of interest, may be used as (or in) the integumental dissolving needle.

Additionally, the present invention may be embodied in an integumental dissolving needle device 1, on which dosage or dosages is printed to clearly indicate how many milligrams of ingredient(s) are present in a given unit area; and which contains grooves or perforations to facilitate the separation of units, or which is pre-sectioned into the corresponding units. Groove(s) may be located on the same side of the device as the needles 4, the opposite side, or both sides. For example, such an embodiment might facilitate the removal of 1 cm² unit(s), each containing 10 mg (or 1.25 mg, etc.) of a pharmaceutical ingredient by sectioning the integumental needle device 1 into 1 cm² units by grooves or perforations, or physically pre-sectioning the device into similar unit(s), and having “10 mg” (“1.25 mg”, etc.) printed on each unit.

### Examples

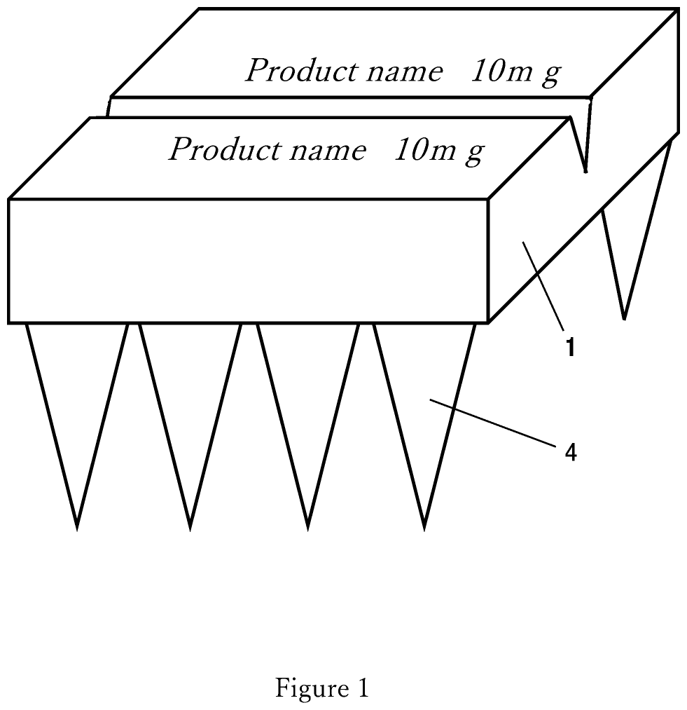

Several examples are depicted below. Possible embodiments of the present invention are not limited to those depicted in FIGS. 1 through 6. For example, the needle device 1 is depicted as a rectangular solid, but other shapes are possible. FIG. 1 is a perspective view of a groove-type integumental (e.g. skin, scales, bark) dissolving needle device 1, with dosage or dosages printed on the surface or surfaces of each section to make explicit how many milligrams of the ingredient are present per unit area. Groove(s) may be located on the same side of the device as the needles 4, the opposite side, or both sides. As a representative example, FIG. 1 depicts such a device in which each section contains 10 mg of ingredient, and in which a single groove is located on the side opposite the needles 4.

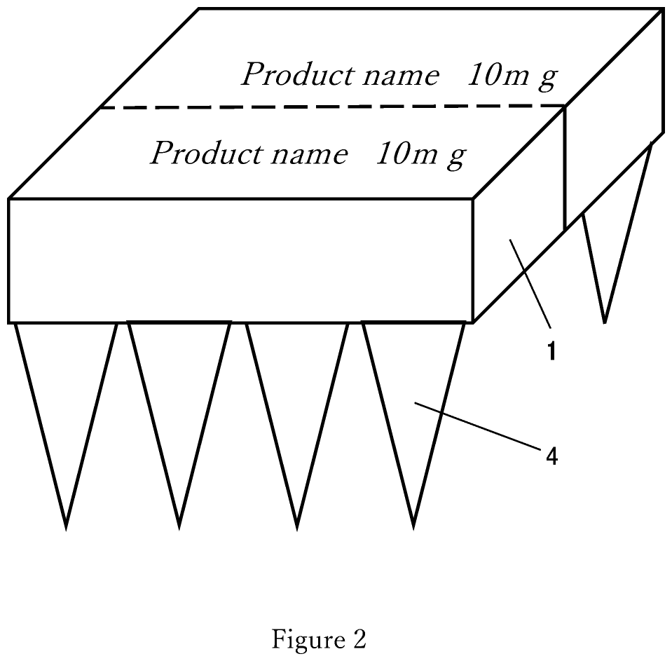

FIG. 2 is a perspective view of a perforation-type integumental (e.g. skin, scales, bark) dissolving needle device 1, with dosage or dosages printed on the surface or surfaces of each section to make explicit how many milligrams of the ingredient are present per unit area. In this example, each section contains 10 mg of ingredient.

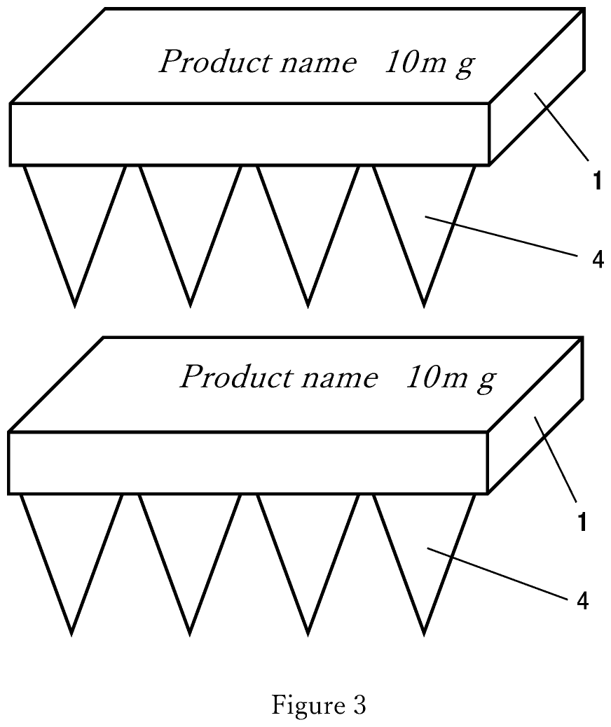

FIG. 3 is a perspective view of a pre-sectioned integumental (e.g. skin, scales, bark) needle device 1, with dosage or dosages printed on the surface or surfaces of each section to make explicit how many milligrams of the ingredient are present per unit area. In this example, each section contains 10 mg of ingredient.

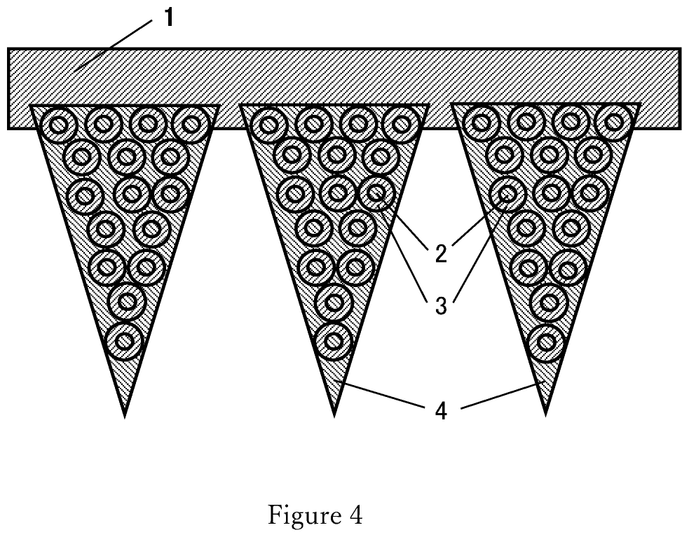

FIG. 4 is a cross-section view of an integumental dissolving needle device 1, in which two-layer granules—consisting of a cosmetic ingredient 2 micronized to a diameter on the micrometer order or smaller (or similarly micronized pharmaceutical ingredient 2), encapsulated by a layer of coating agent 3 that is absorbed into the integument (e.g. skin, scales, bark)—are housed in the integumental (e.g. skin, scales, bark) dissolving needles 4.

FIG. 5 is a cross-section view of an integumental dissolving needle device 1, in which a mixture of two-layer granules and four-layer granules—consisting of a cosmetic ingredient 2 micronized to a diameter on the micrometer order or smaller (or similarly micronized pharmaceutical ingredient 2), encapsulated by a layer of coating agent 3 that is absorbed into the integument (e.g. skin, scales, bark), further coated with an additional layer of micronized cosmetic (or pharmaceutical) ingredient 2, followed by an additional layer of coating agent 3—are housed in the integumental (e.g. skin, scales, bark) dissolving needles 4. As a representative example, FIG. 5 depicts some granules as having a four-layer structure; however, granules may have more than four layers.

FIG. 6 is a cross-section view of an integumental dissolving needle device 1, in which four-layer granules—consisting of a cosmetic ingredient 2 micronized to a diameter on the micrometer order or smaller (or similarly micronized pharmaceutical ingredient 2), encapsulated by a layer of coating agent 3 that is absorbed into the integument (e.g. skin, scales, bark), further coated with an additional layer of micronized cosmetic (or pharmaceutical) ingredient 2, followed by an additional layer of coating agent 3—are housed in the integumental (e.g. skin, scales, bark) dissolving needles 4. As a representative example, FIG. 5 depicts the granules as having a four-layer structure; however, granules may have more than four layers.

### INDUSTRIAL APPLICABILITY

The present invention is not exclusively for use for humans: it may be used for animal and plant species as well, giving it high applicability in veterinary medicine and agriculture industries.

### REFERENCE SIGNS LIST

1. Poultice or patch
2. Micronized pharmaceutical or cosmetic ingredient
3. Coating agent
4. Needle

What is claimed is:

1. An integumental dissolving needle for delivery of one or more pharmaceutical ingredients that are not silk fibroins, or one or more cosmetic ingredients that are not silk fibroins, into skin, scales, bark, or other integumental tissue, characterized by:
the needle housing multi-layer granules, consisting of a base layer of micronized pharmaceutical or cosmetic ingredients, which is encapsulated by a layer of coating agent that is absorbed into the integument, which is covered by another micronized pharmaceutical or cosmetic ingredient layer, which is encapsulated by another coating layer that is absorbed into the integument, repeated for any plural number of ingredient and coating layers.

2. An integumental dissolving needle for delivery of one or more pharmaceutical ingredients that are not silk fibroins, into skin, scales, bark, or other integumental tissue, characterized by:
the needle housing multi-layer granules, consisting of a base layer of micronized pharmaceutical ingredients, which is encapsulated by a layer of coating agent that is absorbed into the integument, which is covered by another micronized pharmaceutical ingredient layer, which is encapsulated by another coating layer that is absorbed into the integument, repeated for any plural number of ingredient and coating layers.

3. An integumental dissolving needle for delivery of one or more cosmetic ingredients that are not silk fibroins, into skin, scales, bark, or other integumental tissue, characterized by:
the needle housing multi-layer granules, consisting of a base layer of micronized cosmetic ingredients, which is encapsulated by a layer of coating agent that is absorbed into the integument, which is covered by another micronized cosmetic ingredient layer, which is encapsulated by another coating layer that is absorbed into the integument, repeated for any plural number of ingredient and coating layers.

4. An integumental dissolving needle for delivery of one or more pharmaceutical ingredients that are not silk fibroins, or one or more cosmetic ingredients that are not silk fibroins, characterized by:
the needle housing multi-layer granules described in claim 1, and additionally granules consisting of a base layer of micronized pharmaceutical or cosmetic ingredients, which is encapsulated by a coating layer that is absorbed into the integument.

5. An integumental dissolving needle for delivery of one or more pharmaceutical ingredients that are not silk fibroins, characterized by:
the needle housing multi-layer granules described in claim 1, and additionally granules consisting of a base layer of micronized pharmaceutical ingredients, which is encapsulated by a coating layer that is absorbed into the integument.

6. An integumental dissolving needle for delivery of one or more cosmetic ingredients that are not silk fibroins, characterized by:
the needle housing multi-layer granules described in claim 1, and additionally granules consisting of a base layer of micronized cosmetic ingredients, which is encapsulated by a coating layer that is absorbed into the integument.

7. An integumental dissolving needle housing the granules described in claim 1, characterized by:
the needle itself being composed of one or more pharmaceutical ingredients that are not silk fibroins, or one or more cosmetic ingredients that are not silk fibroins, or pharmaceutical ingredients encapsulated by a coating layer that is absorbed into the integument, or cosmetic ingredients encapsulated by a coating layer that is absorbed into the integument.

8. An integumental dissolving needle housing the granules described in claim 1, characterized by:
the needle itself being composed of one or more pharmaceutical ingredients that are not silk fibroins, or pharmaceutical ingredients encapsulated by a coating layer that is absorbed into the integument.

9. An integumental dissolving needle housing the granules described in claim 1, characterized by:
the needle itself being composed of one or more cosmetic ingredients that are not silk fibroins, or cosmetic ingredients encapsulated by a coating layer that is absorbed into the integument.

10. An integumental dissolving needle housing the granules described in claim 4, characterized by:
the needle itself being composed of one or more pharmaceutical ingredients that are not silk fibroins, or one or more cosmetic ingredients that are not silk fibroins, or pharmaceutical ingredients encapsulated by a coating layer that is absorbed into the integument, or cosmetic ingredients encapsulated by a coating layer that is absorbed into the integument.

11. An integumental dissolving needle housing the granules described in claim 4, characterized by:
the needle itself being composed of one or more pharmaceutical ingredients that are not silk fibroins, or pharmaceutical ingredients encapsulated by a coating layer that is absorbed into the integument.

12. An integumental dissolving needle housing the granules described in claim 4, characterized by:
the needle itself being composed of one or more cosmetic ingredients that are not silk fibroins, cosmetic ingredients encapsulated by a coating layer that is absorbed into the integument.

13. A needle device consisting of one or more of the integumental dissolving needles described in claim 1, provided on a surface of a poultice or surfaces of a poultice.

14. A needle device consisting of one or more of the integumental dissolving needles described in claim 4, provided on a surface of a poultice or surfaces of a poultice.

15. A needle device consisting of one or more of the integumental dissolving needles described in claim 7, provided on a surface of a poultice or surfaces of a poultice.

16. A needle device consisting of one or more of the integumental dissolving needles described in claim 10, provided on a surface of a poultice or surfaces of a poultice.

17. The integumental dissolving needle or needle device described in claim 1, characterized by:
divisibility, and having a product name or product names and dosage or dosages written on the device surface or surfaces.

18. The integumental dissolving needle or needle device described in claim 4, characterized by:
divisibility, and having a product name or product names and dosage or dosages written on the device surface or surfaces.

19. The integumental dissolving needle or needle device described in claim 10, characterized by:
divisibility, and having a product name or product names and dosage or dosages written on the device surface or surfaces.

20. The integumental dissolving needle or needle device described in claim 14, characterized by:
divisibility, and having a product name or product names and dosage or dosages written on the device surface or surfaces.
